(19) United States
(12) Patent Application Publication
(10) Pub. No.: US 2005/0281750 A1
(43) Pub. Date: Dec. 22, 2005

(54) Sprayable compositions comprising a combination of pharmaceutical active agents, an alcohol phase, at least one volatile silicone and a non-volatile oily phase

(75) Inventors: Nathalie Willcox, Saint Vallier De Thiey (FR); Sandrine Orsoni, Mandelieu (FR); Claire Mallard, Mougins Le Haut (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: GALDERMA S.A.

(21) Appl. No.: 10/958,236

(22) Filed: Oct. 6, 2004

(30) Foreign Application Priority Data
Jun. 17, 2004 (FR) 04/06614

(51) Int. Cl. A61L 9/04; A61K 31/59

(52) U.S. Cl. 424/45; 514/167; 514/171

(57) ABSTRACT

Sprayable, anhydrous and physically/chemically stable dermatological/pharmaceutical compositions, well suited for the treatment of a variety of dermatological disorders, notably psoriasis, contain: a) a therapeutically effective amount of a solubilized corticord; notably clobetasol propionate; b) a therapeutically effective amount of a solubilized vitamin D derivative, notably calcitriol; c) an alcohol phase; d) at least one volatile silicone; and e) a non-volatile oily phase which comprises one or more oils; formulated into f), a sprayable and topically applicable, dermatologically/pharmaceutically acceptable vehicle therefor.

### CROSS-REFERENCE TO PRIORITY APPLICATION

This application claims priority under 35 U.S.C. § 119 of FR 04/06614, filed Jun. 17, 2004, hereby expressly incorporated by reference and assigned to the assignee hereof.

### BACKGROUND OF THE INVENTION

1. Technical Field of the Invention

The present invention relates to anhydrous compositions in the form of a spray comprising a combination of clobetasol propionate (corticoid) and calcitriol (vitamin D derivative) as pharmaceutical active ingredient, an alcohol phase, at least one volatile silicone and a non-volatile oily phase in a physiologically acceptable medium, to the process for preparing same and to cosmetics and dermatology applications thereof. The compositions afford a good penetration of the active ingredient through the cutaneous layers.

2. Description of Background and/or Related and/or Prior Art:

It is not conventional to use a combination of active principles in the treatment of dermatological complaints. The main difficulties encountered by those skilled in the art when combining two active principles are the problems of chemical instability and the interactions which the active principles may undergo when they are present in the same formulation.

Few treatments therefore exist which combine calcitriol and a corticoid. In fact, vitamin D and its derivatives are unstable in aqueous media and sensitive to acidic pH values, whereas corticoids, and more particularly clobetasol propionate, are sensitive to basic media. It was not therefore obvious to those skilled in the art to combine and stabilize an active ingredient of the vitamin D type and a corticosteroid in one and the same composition.

Calcitriol is a vitamin D analogue used to regulate the calcium level in the organism. Its use in the treatment of dermatological diseases has been described especially in U.S. Pat. No. 4,610,978 for the treatment of psoriasis. Said patent suggests compositions comprising calcitriol that can also contain an amount of an anti-inflammatory such as a corticosteroid, but no concrete embodiment of a combination of calcitriol and a corticosteroid is either described or tested in terms of efficacy.

FR-2,848,454, assigned to the assignee hereof, describes that a combination of calcitriol with a corticosteroid made it possible to obtain a synergistic effect in the treatment of certain dermatological complaints such as psoriasis, atopic dermatitis, contact dermatitis and seborrhoeic dermatitis, without however proposing stable pharmaceutical compositions combining both active ingredients.

Furthermore, in the field of dermatology and the formulation of pharmaceutical compositions, those skilled in the art are induced to seek compositions which not only have to be physically and chemically stable, but also have to make it possible to release the active ingredient and promote its penetration through the cutaneous layers so as to improve its efficacy.

The pharmaceutical compositions moreover have to have a good cosmetic character and preferably be non-irritant.

There are currently numerous topical compositions that comprise an active ingredient and are capable of promoting its penetration into the skin by virtue of the presence especially of a high content of propenetrating glycol. These compositions are formulated as emulsions with a high content of fatty phase, commonly called “lipocreams”, as anhydrous compositions called “unguents”, as fluid compositions with a high content of volatile solvents such as ethanol or isopropanol, intended for application to the scalp and also called “hair lotions”, or as viscous O/W emulsions, also called “O/W creams”.

The stabilization of a formulation comprising such a percentage of glycol makes it necessary to use, in the emulsion, emulsifiers and stabilizers of the glyceryl stearate or PEG 100 stearate type, or stabilizers or consistency factors of the white wax or cetostearyl alcohol type, which give rise to the formation of a viscous cream, i.e., a cream with a viscosity greater than 10 Pa·s (10,000 centipoises, measured with a Brookfield LVDV II apparatus+no. 4 cup, at a speed of 30 rpm for 30 seconds and at a temperature of 25° C.±3° C.). This viscosity therefore makes the product difficult to apply. Hence, these compositions, on the one hand, have a poor cosmetic acceptability due to their viscosity, and, on the other hand, carry risks of intolerance caused by the presence of high proportions of glycol. In addition, these high viscosities make the formulations difficult to apply to the different parts of the body affected by the pathological condition. Consequently, the majority of existing treatments, in the form of creams, gels or ointments, require the help of a third party to apply them to the areas that are difficult to reach. The third party therefore has to touch both the product containing the active ingredient and the psoriatic plaques, resulting in a situation that is not ideal from the point of view of the comfort of the user and the safety of the third party. Those skilled in the art are also aware that non-compliance with the prescribed treatment for reasons referred to above is one of the main causes of treatment failure, the article “Patients with psoriasis and their compliance with medication” (Richards et al., J. Am. Acad. Dermatol., October 1999, pp 581-583) indicating that nearly 40% of patients with a chronic disease like psoriasis do not follow their treatment. It has been demonstrated that the patient's compliance with his treatment is directly related to the characteristics of the vehicle of the composition applied. The article “Patients with psoriasis prefer solution and foam vehicles: a quantitative assessment of vehicle preference” (Housman et al., CUTIS, December 2002, Vol. 70, pp 327 to 332) indicates that psoriasis patients prefer a solution or a foam to an unguent, a cream or a gel.

Those skilled in the art are furthermore familiar with formulations containing silicone compounds which result in compositions that are pleasant to use. Thus, in U.S. Pat. No. 6,538,039, a novel formulation of active ingredient for transdermal administration was developed which comprises silicone compounds for depositing a film on the surface of the skin. Also in said patent, the transdermal passage is facilitated by the obligatory presence of an absorption promoter, glycols being among the other compounds mentioned.

In EP-0,966,972, the compositions described can be formulated as a spray and they comprise an active compound, a silicone gum and a pharmaceutically acceptable excipient. The problem which the invention described in EP-0,966,972 sets out to solve is to deposit a substantive film on the surface of the skin, said problem being solved by the presence of the silicone gum.

The problem which the present invention described hereinbelow solves are physically and chemically stable compositions that allow calcitriol and clobetasol propionate (or clobetasol 17-propionate, these two names being used indiscriminately hereafter) to be combined in one and the same formulation. These compositions improve the penetration of the pharmaceutical active ingredients while avoiding the presence of a high glycol content. The compositions according to the invention are also easy to use and have an acceptable cosmetic character for application to all areas of the body that may be affected by the pathological condition, disorder or affliction.

The prior art closest to the invention is WO 00/64450, which indicates the use of a pharmaceutical composition containing a vitamin D analogue and a corticosteroid. All of the composition examples in said patent application combine solely calcipotriol and betamethasone dipropionate. The preferred compositions described in the patent application that make it possible to stabilize the two active ingredients are compositions in the form of an unguent. However, these compositions exhibit the abovementioned disadvantages as regards comfort and ease of application. Study of this prior art in no way suggests sprayable, i.e., easily applicable, compositions such as those described in the present patent application with the active ingredients clobetasol propionate and calcitriol, which are solubilized and stable in the composition.

### SUMMARY OF THE INVENTION

The problem which the present invention solves are physically and chemically stable compositions that allow the two active ingredients calcitriol and clobetasol propionate to be combined in one and the same composition, said ingredients acting synergistically for the treatment of psoriasis, the compositions according to the invention also being easy to use and having an acceptable cosmetic character for application to all areas of the body that may be affected by the pathological condition.

“Physical stability” is understood according to the invention as applying to a composition that does not undergo any modification of macroscopic appearance (phase separation, change of color or appearance, etc.) or microscopic appearance (recrystallization of active ingredients) after storage at temperatures of 4° C. and 40° C. for 2, 4, 8 and 12 weeks.

“Chemical stability” is understood according to the invention as applying to a composition in which the active principle content remains stable after three months at room temperature and at 40° C. A stable active principle content means according to the invention that the content varies very little relative to the initial content, i.e., that the variation in active principle content at time T must not be less than 90% of the initial content at T0 and preferably not less than 95% of the initial content at T0.

Thus, it has now surprisingly been found that compositions comprising the following, formulated into a pharmaceutically acceptable vehicle therefor:
a) a therapeutically effective amount of a corticoid in solubilized form, and more particularly clobetasol propionate;
b) a therapeutically effective amount of a vitamin D derivative in solubilized form, and more particularly calcitriol;
c) an alcohol;
d) at least one volatile silicone; and
e) a non-volatile oily phase which comprises one or more oils,
   said compositions being sprayable, i.e., suitable for packaging in the form of a spray, are compositions which ameliorate or avoid the above disadvantages and drawbacks of the prior art.

While allowing a good penetration of the active principles, the compositions of the present invention also have a very good patient acceptability and tolerance, as described in the examples of the present invention. The compositions according to the invention are therefore found to be particularly suitable for the treatment of dermatological complaints and more particularly suitable for the treatment of psoriasis.

The present invention therefore features sprayable compositions comprising the following, in a pharmaceutically acceptable vehicle:
a) a therapeutically effective amount of clobetasol propionate in solubilized form;
b) a therapeutically effective amount of calcitriol in solubilized form;
c) an alcohol phase;
d) at least one volatile silicone; and
e) a non-volatile oily phase which comprises one or more oils.

### DETAILED DESCRIPTION OF BEST MODE AND SPECIFIC/PREFERRED EMBODIMENTS OF THE INVENTION

Advantageously, the compositions according to the invention comprise from 0.00001 to 0.1% by weight, preferably from 0.0001 to 0.001% by weight and particularly preferably from 0.0002 to 0.0005% by weight of an active ingredient derived from vitamin D, based on the total weight of the composition. The compositions according to the invention comprise more particularly 0.0003% by weight of calcitriol, based on the total weight of the composition.

Advantageously, the compositions according to the invention comprise from 0.0001 to 0.1% by weight and preferably from 0.001 to 0.05% by weight of a corticoid, based on the total weight of the composition. The preferred compositions according to the invention comprise more particularly 0.025% or 0.05% by weight of clobetasol propionate, based on the total weight of the composition.

“Alcohol phase” is understood according to the invention as meaning at least one alcohol compound. Non-limiting examples which may be mentioned of alcohols usable according to the invention are linear or branched aliphatic alcohols such as anhydrous ethanol, isopropanol and butanol. The compositions according to the invention preferably contain ethanol. Advantageously, the compositions contain from 5 to 60% by weight and preferably from 10 to 40% by weight of an alcohol, based on the total weight of the composition.

A preferred composition according to the invention contains from 10 to 40% by weight of ethanol.

“Volatile silicones” are understood according to the invention as meaning polyorganosiloxane compounds which can be cyclic or linear and have a measurable pressure under ambient conditions. The cyclic volatile silicones according to the invention are polydimethylcyclosiloxanes, namely compounds of the formula: 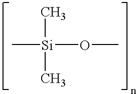
where n has an average value of from 3 and 6 and is preferably 4 or 5, which are generally known as cyclomethicones. The linear volatile silicones according to the invention are linear polysiloxanes such as hexamethyldisiloxane or low-molecular weight dimethicones. The linear volatile silicones generally have a viscosity below about 5 centistokes at 25° Celsius, while the cyclic volatile silicones have a viscosity below about 10 centistokes at 25° Celsius.

The preferred volatile silicones according to the invention are linear siloxanes, particularly preferably hexamethyldisiloxane. The product DC Fluid 0.65 cSt marketed by DOW CORNING may be mentioned as an example.

Advantageously, the compositions according to the invention comprise from 10 to 60% by weight, preferably from 20 to 50% by weight and particularly preferably from 15 to 45% by weight of volatile silicone, based on the total weight of the composition.

“Non-volatile oily phase” is understood according to the invention as meaning a non-volatile oily phase that is appropriate for a pharmaceutical or cosmetic composition. Non-volatile oils generally have a viscosity above about 10 centipoises at 25° C. and can reach a viscosity ranging up to 1,000,000 centipoises at 25° C. The non-volatile oil can be one of a wide variety of synthetic or natural silicone or organic oils, a non-exhaustive list of which is given by way of indication.

(a) Esters:

Examples of non-volatile oils usable according to the invention comprise esters of the formula RCO—OR′, where R and R′, which are identical or different, are a linear or branched alkyl, alkenyl, alkoxycarbonylalkyl or alkoxycarbonyloxyalkyl chain having from 1 to 25 carbon atoms and preferably from 4 to 20 carbon atoms. Examples of such esters include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coconut dicaprylate/caprate, decyl isostearate, isodecyl oleate, isodecyl neopentanoate, isohexyl neopentanoate, octyl palmitate, dioctyl malate, tridecyl octanoate, myristyl myristate and octyldodecanol.
(b) Fatty Acid Glyceryl Esters:

The oil can also comprise fatty esters of natural fatty acids, or triglycerides of animal or vegetable origin. Examples of these include castor oil, lanolin oil, triisocetyl citrate, triglycerides having from 10 to 18 carbon atoms, caprylic/capric triglycerides, coconut oil, maize oil, cottonseed oil, linseed oil, mink oil, olive oil, palm oil, mahua butter, colza oil, soya oil, sunflower oil, nut oil and equivalent compounds.

(c) Fatty Acid Glycerides:

Other suitable oils are synthetic or semisynthetic glyceryl esters such as fatty acid mono-, di- and triglycerides, which are modified natural oils or fats, for example glyceryl stearate, glyceryl dioleate, glyceryl distearate, glyceryl trioctanoate, glyceryl linoleate, glyceryl myristate, glyceryl isostearate, PEG castor oils, PEG glyceryl oleates, PEG glyceryl stearates and equivalent compounds.

(d) Non-Volatile Hydrocarbons:

Other very suitable non-volatile solvents for the compositions according to the invention are non-volatile hydrocarbons such as paraffins, isoparaffins, mineral oils and equivalent compounds.

(e) Guerbet Esters:

Guerbet esters are esters resulting from the reaction of a Guerbet alcohol of the general formula: 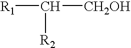
with a carboxylic acid of the general formula:
R₃COOH or HOOC—R₃—COOH,
in which R₁ and R₂, which are identical or different, are an alkyl having from 4 to 20 carbon atoms and R₃ is a substituted or unsubstituted fatty radical such as a saturated or unsaturated, linear or branched alkyl or alkylene chain having from 1 to 50 carbon atoms, or a phenyl capable of being substituted by a halogen, a hydroxyl, a carboxyl or an alkylcarbonylhydroxyl.

The Guerbet alcohols mentioned above, especially those of the octyldodecanol type marketed under the name Eutanol G, are also suitable for the compositions according to the invention.

Preferably, the compounds constituting the oily phase of the compositions according to the invention are the caprylic/capric triglycerides marketed under the name Miglyol 812, the cetearyl isononanoate marketed under the name Cetiol SN, and vegetable oils (sweet-almond oil, sesame oil, wheatgerm oil, olive oil, jojoba oil, etc.), used by themselves or in a mixture.

The compositions according to the invention will preferably comprise Miglyol 812. In fact, triglycerides are one of the components of the skin and form part of the skin's natural lipids together with ceramides, cholesterol and phospholipids. They integrate into the deep layers of the epidermis and compensate for the skin's loss of hydration. The skin's protective barrier is regenerated in a specific and durable manner.

Medium chain triglycerides, including the Miglyol 812 used, are caprylic (C8) and capric (C10) fatty acid compounds derived from coconut oil or palm kernel oil.

Its main properties are as follows:
it is a low viscosity emollient that enhances spreading over the skin,
it is a solvent for lipophilic active ingredients, penetrates the skin rapidly and promotes the penetration of active ingredients, and
it does not impart a greasy sensation on application and does not leave greasy residues.

It is particularly recommended to use emollients in the case of a pathological condition such as psoriasis. Emollients help to hydrate and soothe psoriatic plaques.

If, for example, a high concentration of alcohol or volatile solvent is applied, burning sensations can disturb patients with highly irritated skin.

To compensate for these sensations of irritation, the use of lipids as emollients makes it possible to restore the normal function of the cutaneous barrier and protect it from external influences.

Advantageously, the compositions according to the invention comprise from 1 to 80% by weight, preferably from 20 to 60% by weight and particularly preferably from 30 to 45% by weight of oily phase, based on the total weight.

In one preferred embodiment, the compositions according to the invention also contain antioxidant compounds such as DL-a-tocopherol, butylhydroxyanisole or butylhydroxytoluene, propyl gallate, superoxide dismutase, ubiquinol or certain metal chelating agents. The antioxidants preferably used in the compositions according to the invention are DL-a-tocopherol, butylhydroxyanisole and butylhydroxytoluene.

In one preferred embodiment of a composition according to the invention, the composition also comprises a silicone gum. It has also, surprisingly, now been determined that a composition comprising a silicone gum in the concentrations defined below enables the active ingredient to penetrate more rapidly through the various cutaneous layers.

“Silicone gums” are understood as meaning silicone gums known to those skilled in the art, especially the ones described in EP-0,966,972, which is incorporated here by way of reference. In this preferred embodiment of a composition according to the invention, the silicone gum is introduced at a concentration of from 0.001 to 3% by weight and preferably of from 0.01 to 1% by weight. Dow Corning offers a commercial product sold under the name DC Silmogen Carrier, which is composed of 99% of hexamethyldisiloxane and 1% of silicone gum, said product advantageously being usable in one of the compositions according to the invention.

The compositions according to the invention can also contain surfactants. The surfactants usable according to the invention are of the anionic surfactant type such as carboxylates and especially soaps, alkylarylsulfonates, alkylethersulfates, alkylsulfates and alcohol sulfates. More particularly, the anions of these surfactants are coupled with a cation such as that of the metal sodium or potassium. Other preferred surfactants according to the invention are those of the polysorbate and poloxamer types.

Preferably, the surfactants used according to the present invention are sodium laurylsulfate, polysorbate 80 (TWEEN 80 from Uniqema) and poloxamer 124 (SYNPERONIC PEL44 from Uniqema).

The pharmaceutically acceptable vehicle according to the invention must be chosen in such a way that the advantageous properties intrinsically associated with the present invention are unaffected or substantially unaffected by the envisaged addition. The vehicle can be composed of a single excipient such as a solvent, or a mixture of excipients such as those used to formulate an emulsion. Non-limiting examples which may be mentioned of excipients that can be used, by themselves or in a mixture, are water, solvents, diluents, or any excipient that can be used to formulate an emulsion, a milk, a gel, an unguent or a foaming composition. These excipients are compounds commonly used in the formulation of pharmaceutical compositions. Preferably, the excipients according to the invention are water, alcohols, polyols, ethers, esters, aldehydes, ketones, fatty acids and alcohols, and fatty esters. Particularly preferably, the excipient used will be an alcohol such as ethanol.

In one particular embodiment, the composition according to the invention comprises the following, in a pharmaceutically acceptable vehicle:
a) from 0.0001 to 0.1% of clobetasol propionate;
b) from 0.00001 to 0.1% of calcitriol;
c) from 5 to 60% of ethanol;
d) from 10 to 60% of volatile silicone; and
e) from 1 to 80% of an oily phase which comprises one or more oils selected from among caprylic/capric triglycerides, cetearyl isononanoate and vegetable oils.

In one preferred embodiment, the composition according to the invention comprises the following, in a pharmaceutically acceptable vehicle:
a) from 0.001 to 0.05% of clobetasol propionate;
b) from 0.0002 to 0.0005% of calcitriol;
c) from 10 to 40% of ethanol;
d) from 15 to 45% of volatile silicone; and
e) from 30 to 45% of an oily phase which comprises one or more oils selected from among caprylic/capric triglycerides, cetearyl isononanoate and vegetable oils.

The preferred compositions according to the invention are therefore sprayable compositions comprising the following, in a pharmaceutically acceptable vehicle:
a) a therapeutically effective amount of clobetasol propionate in solubilized form;
b) a therapeutically effective amount of calcitriol in solubilized form;
c) an alcohol phase;
d) at least one volatile silicone;
e) a non-volatile oily phase which comprises one or more oils;
g) an antioxidant; and
h) a silicone gum.

The pharmaceutical compositions according to the invention may also contain inert additives or combinations of these additives, such as
wetting agents;
flavor improvers;
preservatives such as parahydroxybenzoic acid esters;
stabilizers;
humidity regulators;
pH regulators;
osmotic pressure modifiers;
emulsifiers;
UV-A and UV-B filters;
propenetrating agents; and
synthetic polymers.

Of course, those skilled in the art will take care to choose any compound(s) to be added to these compositions in such a way that the advantageous properties intrinsically associated with the present invention are unaffected or substantially unaffected by the envisaged addition.

The compositions according to the invention are more particularly suited for a regime or regimen for the treatment of skin and mucosae; same are sprayable and suitable for packaging in the form of a spray.

The spray can be obtained by conventional formulating means known to those skilled in the art. For example, the composition can be sprayed by a mechanical sprayer which pumps the composition from a container, bottle or equivalent vessel. Likewise, the composition can be propelled by means of a gas in the manner well known to those skilled in the art. The conventional propellant gases, such as air or hydrocarbons, are effective provided they do not interfere with the composition. The composition passes through a nozzle, which can be pointed directly at the desired application site. The nozzle can be chosen so as to apply the composition in the form of a vapor or a jet of droplets according to the techniques known to those skilled in the art. Depending on the chosen pharmaceutical active ingredient, the spraying mechanism must be capable always of dispensing the same amount of active ingredient. The mechanisms for controlling the amount of composition to be dispensed by the spray are also known to those skilled in the art. For example, the amount of propellant gas can be calculated so as to propel the exact amount of product desired.

The compositions according to the invention will preferably be dispensed using a dosing vaporizer bottle whose characteristics of application area and dose are controlled and reproducible. For example, the vaporizer used consists of a bottle equipped with a 25 μl dosing valve.

The present invention further relates to the formulation of a composition according to the invention for the preparation of a drug suited for the treatment of:
dermatological conditions or afflictions associated with a keratinization disorder related to differentiation and proliferation, especially acne vulgaris, black heads, polymorphous acne, acne rosacea, nodulocystic acne, acne conglobata, senile acne, and secondary acne such as solar acne, acne medicamentosa or occupational acne;
ichthyosis, ichthyosiform conditions, Darrier's disease, palmoplantar keratoderma, leukoplakia and leukoplakiform states, and cutaneous or mucous (buccal) lichen;
dermatological disorders having an inflammatory immunoallergic component and with or without cellular proliferation disorder, especially cutaneous, mucous or ungueal psoriasis, psoriatic rheumatism, and cutaneous atopy such as eczema, respiratory atopy or gingival hypertrophy;
benign or malignant dermal or epidermal proliferations of viral or non-viral origin, especially verrucas, plane warts, epidermodysplasia verruciformis, oral or florid papillomatosis, and T lymphoma;
proliferations inducible by ultraviolet, especially basal cell and spinal cell epithelioma;
precancerous cutaneous lesions, especially keratoacanthomas;
immune dermatoses, especially lupus erythematosus;
bullous immune diseases;
collagen diseases, especially scleroderma;
dermatological or systemic disorders having an immunological component;
cutaneous disorders due to exposure to UV radiation, photoinduced or chronological aging of the skin, or actinic pigmentations and keratoses, or any pathological conditions associated with chronological or actinic aging, especially xerosis;
sebaceous function disorders, especially hyperseborrhoeic acne, simple seborrhoea or seborrhoeic dermatitis;
healing or cicatrization disorders or striae atrophicae;
pigmentation disorders such as hyperpigmentation, melasma, hypopigmentation or vitiligo;
disorders of the lipid metabolism, such as obesity, hyperlipidaemia, non-insulin-dependent diabetes or syndrome X;
inflammatory afflictions such as arthritis;
cancerous or precancerous states;
alopecia of different origins, especially that due to chemotherapy or radiation;
immune system disorders such as asthma, type I sugar diabetes, multiple sclerosis or other selective dysfunctions of the immune system; or
disorders of the cardiovascular system, such as arteriosclerosis or hypertension.

In particular, the invention relates to the formulation of a composition as defined above for the preparation of a pharmaceutical composition suited for the treatment of psoriasis.

In one preferred embodiment of the composition, it will contain 0.025% of clobetasol 17-propionate and 0.0003% of calcitriol in the presence of ethanol, and will be used for the preparation of a drug intended for the treatment of psoriasis.

In another preferred embodiment of the invention, the ratio volatile solvent/non-volatile phase is optimized so as to retain a rapid solvent evaporation favorable to the penetration of the active ingredients, but so as to provide a sufficient proportion of non-volatile phase to give the composition substantivity and thus enable the product to stay longer on the skin, favoring the emollience.

The ratio volatile solvent/non-volatile phase will preferably range from 1 to 2.5.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Test for Stability of Calcitriol in Different Oily and/or Alcoholic Phases

Calcitriol stability data were generated in various excipients, including ethanol 100, an ethanol 100 (75%)/cyclomethicone 5 (25%) mixture, or oils such as Miglyol 812 and Cetiol SN.
a) Stability of Calcitriol in Ethanol:

Solution of 30 ppm of calcitriol in qsp 100% of absolute ethanol, in the presence of 0.02% of BHT.

Technique of HPLC assay against a reference substance.

At the starting time (T0) the composition is considered to comprise 100% of calcitriol.

Measured concentration of calcitriol in % relative to T0:

| Stability | T 1 | T 2 | T 3 | T 4 |
|---|---|---|---|---|
| conditions | week | weeks | weeks | weeks |
| −18° C. | 100.9% | 100.5% | 99.5% | 99.5% |
| +4° C. | 97.7% | 98.6% | 98.1% | 97.7% |
| +30° C. | / | 93.4% | / | 93.0% |

These results show that calcitriol has a good stability in ethanol.

b) Stability of Calcitriol in Ethanol/Cyclopentasiloxane:

Solution of 30 ppm of calcitriol in qsp 75% of absolute ethanol+25% of cyclopentasiloxane, in the presence of 0.02% of BHT.

Technique of HPLC assay against a reference substance.

At the starting time (T0) the composition is considered to comprise 100% of calcitriol.

Measured concentration of calcitriol in % relative to T0:

| Stability | | | |
|---|---|---|---|
| conditions | T 2 weeks | T 3 weeks | T 4 weeks |
| −18° C. | 100.4% | 101.3% | 99.2% |
| +4° C. | 99.2% | 99.6% | 97.5% |
| +30° C. | 93.8% | / | 93.3% |

These results show that calcitriol has a good stability in an ethanol/cyclopentasiloxane mixture.

c) Stability of Calcitriol in Miglyol 812 (Caprylic/Capric Triglycerides):

Solution of 30 ppm of calcitriol in qsp 100% of Miglyol 812, in the presence of 0.4% of BHT.

Technique of HPLC assay against a reference substance.

At the starting time (T0) the composition is considered to comprise 100% of calcitriol.

Measured concentration of calcitriol in % relative to T0:

| Stability conditions | T 2 weeks | T 4 weeks |
|---|---|---|
| +4° C. | 98.3% | 105.2% |
| RT | 95.1% | 98.0% |
| +40° C. | 91% | 93.8% |

These results show that calcitriol has a good stability in Miglyol 812.

d) Stability of Calcitriol in Cetiol SN (Cetearyl Isononanoate):

Solution of 30 ppm of calcitriol in qsp 100% of Cetiol SN (cetearyl isononanoate), in the presence of 0.4% of BHT.

Technique of HPLC assay against a reference substance.

At the starting time (T0) the composition is considered to comprise 100% of calcitriol.

Measured concentration of calcitriol in % relative to T0:

| Stability conditions | T 2 weeks | T 4 weeks |
|---|---|---|
| +4° C. | 98.6% | 98.1% |
| RT | 98.7% | 98.4% |
| +40° C. | 99.0% | 98.9% |

These results show that calcitriol has a good stability in Cetiol SN.

### EXAMPLE 2

### Process for the Preparation of the Compositions of the Examples Below

The compositions according to the invention are prepared at room temperature, under a hood and in inactinic light.

The antioxidant, the calcitriol and the alcohol are introduced into a flask and stirred until the calcitriol is perfectly solubilized.

The clobetasol propionate is then added and stirring is continued until the clobetasol propionate is solubilized.

When the two active ingredients are perfectly solubilized, the remaining constituents of the formulation are introduced in succession.

The mixture is stirred until it is perfectly homogeneous.

### EXAMPLE 3:

| CONSTITUENT | % |
|---|---|
| 2-PROPANOL | qs 100 |
| DL-ALPHA-TOCOPHEROL | 0.05 |
| CALCITRIOL | 0.0003 |
| CLOBETASOL 17-PROPIONATE | 0.001 |
| CETEARYL ISONONANOATE | 40 |
| 1,2-PROPANEDIOL | 10 |
| DIMETHICONOL AND | 35 |
| HEXAMETHYLDISILOXANE | |

The procedure is as described in Example 2 above.

A colorless liquid solution is obtained.

### EXAMPLE 4:

a) Composition:

| CONSTITUENT | % |
|---|---|
| ABSOLUTE ETHANOL | qs 100 |
| BUTYLHYDROXYTOLUENE | 0.04 |
| CALCITRIOL | 0.0009 |
| CLOBETASOL 17-PROPIONATE | 0.025 |
| MEDIUM CHAIN TRIGLYCERIDES | 30 |
| DIMETHICONOL AND | 40 |
| HEXAMETHYLDISILOXANE | |

The procedure is as described in Example 2 above.

A colorless liquid solution is obtained.

b) Physical Stability of the Composition:

The physical stability of the formulations is measured by macroscopic and microscopic observation of the formulation at room temperature and at 4° C. after 2, 4, 8 and 12 weeks. At room temperature, macroscopic observation makes it possible to guarantee the physical integrity of the products and microscopic observation makes it possible to verify that there is no recrystallization of the solubilized active ingredients.

Non-recrystallization of the solubilized active ingredients is verified by microscopic observation at 4° C.

Specifications at T0:

Macroscopic appearance: colorless liquid solution

Microscopic appearance: absence of crystals of calcitriol and clobetasol 17-propionate

Time

| Stability conditions | T 15 days | T 1 month |
|---|---|---|
| RT | Conforms | Conforms |
| +4° C. | Conforms | Conforms |

c) Chemical Stability of the Composition:

Stability of the Calcitriol:

Assay of the active ingredient by external calibration using HPLC.

The results are expressed in % recovery relative to the theoretical value.

| Stability | Time | | |
|---|---|---|---|
| conditions | T 15 days | T 1 month | T 2 months |
| RT | 101.2 | 101.6 | 100.8 |

Stability of the Clobetasol 17-Propionate:

Assay of the active ingredient by internal calibration using HPLC.

The results are expressed in % recovery relative to the theoretical value.

| Stability | Time | | |
|---|---|---|---|
| conditions | T 15 days | T 1 month | T 2 months |
| RT | 102.4 | 103.2 | 101.4 |

### EXAMPLE 5:

a) Composition:

| CONSTITUENT | % |
|---|---|
| ABSOLUTE ETHANOL | qs 100 |
| BUTYLHYDROXYTOLUENE | 0.04 |
| CALCITRIOL | 0.0009 |
| CLOBETASOL 17-PROPIONATE | 0.025 |
| MEDIUM CHAIN TRIGLYCERIDES | 45 |
| DIMETHICONOL AND | 25 |
| HEXAMETHYLDISILOXANE | |

The procedure is as described in Example 2 above.

A colorless liquid solution is obtained.

b) Physical Stability of the Composition:

Specifications at T0:

Macroscopic appearance: colorless liquid solution

Microscopic appearance: absence of crystals of calcitriol and clobetasol 17-propionate

Time

| Stability conditions | T 15 days | T 1 month |
|---|---|---|
| RT | Conforms | Conforms |
| +4° C. | Conforms | Conforms |

c) Chemical Stability of the Composition:

Stability of the Calcitriol:

Assay of the active ingredient by external calibration using HPLC.

The results are expressed in % recovery relative to the theoretical value.

Time

| Stability conditions | T 15 days | T 1 month |
|---|---|---|
| RT | 103.3 | 101.2 |

Stability of the Clobetasol 17-Propionate:

Assay of the active ingredient by internal calibration using HPLC.

The results are expressed in % recovery relative to the theoretical value.

Time

| Stability conditions | T 15 days | T 1 month |
|---|---|---|
| RT | 101.5 | 102.7 |

### EXAMPLE 6:

a) Composition:

| CONSTITUENT | % |
|---|---|
| ABSOLUTE ETHANOL | qs 100 |
| BUTYLHYDROXYTOLUENE | 0.04 |
| CALCITRIOL | 0.0003 |
| CLOBETASOL 17-PROPIONATE | 0.025 |
| MEDIUM CHAIN TRIGLYCERIDES | 45 |
| DIMETHICONOL AND | 25 |
| HEXAMETHYLDISILOXANE | |

The procedure is as described in Example 2 above.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. A sprayable, anhydrous and physically/chemically stable dermatological/pharmaceutical composition, comprising:
a) a therapeutically effective amount of a solubilized corticord;
b) a therapeutically effective amount of a solubilized vitamin D derivative;
c) an alcohol phase;
d) at least one volatile silicone; and
e) a non-volatile oily phase which comprises one or more oils; formulated into f), a sprayable and topically applicable,
dermatologically/pharmaceutically acceptable vehicle therefor.

2. The sprayable, anhydrous dermatological/pharmaceutical composition as defined by claim 1, said corticoid comprising clobetasol propionate.

3. The sprayable, an hydrous dermatological/pharmaceutical composition as defined by claim 2, said vitamin D derivative comprising calcitriol.

4. The sprayable, anhydrous dermatological/pharmaceutical composition as defined by claim 1, said alcohol phase comprising ethanol.

5. The sprayable, anhydrous dermatological/pharmaceutical composition as defined by claim 1, said at least one volatile silicone comprising hexamethyldisiloxane.

6. The sprayable, anhydrous dermatological/pharmaceutical composition as defined by claim 1, said oily phase comprising one or more oils selected from the group consisting of caprylic/capric triglycerides, cetearyl isononanoate and vegetable oils.

7. The sprayable, anhydrous dermatological/pharmaceutical composition as defined by claim 4, comprising:
a) from 0.001 to 0.05% of clobetasol propionate;
b) from 0.0002 to 0.0005% of calcitriol;
c) from 10 to 40% of ethanol;
d) from 15 to 45% of at least one volatile silicone; and
e) from 30 to 45% of an oily phase which comprises one or more oils selected from the group consisting of caprylic/capric triglycerides, cetearyl isononanoate and vegetable oils.

8. The sprayable, anhydrous dermatological/pharmaceutical composition as defined by claim 1, further comprising an antioxidant compound.

9. The sprayable, anhydrous dermatological/pharmaceutical composition as defined by claim 8, said antioxidant being selected from the group consisting of DL-a-tocopherol, butylhydroxyanisole and butylhydroxytoluene.

10. The sprayable, anhydrous dermatological/pharmaceutical composition as defined by claim 1, further comprising a silicone gum.

11. The sprayable, anhydrous dermatological/pharmaceutical composition as defined by claim 10, comprising
a) a therapeutically effective amount of solubilized clobetasol propionate;
b) a therapeutically effective amount of solubilized calcitriol;
c) an alcohol phase;
d) at least one volatile silicone;
e) a non-volatile oily phase which comprises one or more oils;
g) an antioxidant; and
h) a silicone gum.

12. The sprayable, anhydrous dermatological/pharmaceutical composition as defined by claim 1, further comprising a surfactant compound.

13. The sprayable, anhydrous dermatological/pharmaceutical composition as defined by claim 12, said surfactant being selected from the group consisting of sodium laurylsulfate, poloxamers and polysorbates.

14. A regime or regimen for preventing or treating dermatological conditions associated with a keratinization disorder relating to differentiation and to proliferation, common acne, comedo-type acne, polymorphic acne, acne rosacea, nodulocystic acne, acne conglobata, senile acne, secondary acne, solar acne, drug-induced acne or occupational acne; ichthyoses, ichthyosiform conditions, Darrier's disease, palmoplantar keratodermas, leukoplakia and leukoplakiform conditions, cutaneous lichen or mucosal (oral) lichen; dermatological conditions having an inflammatory immunoallergic component, with or without a cell proliferation disorder, cutaneous psoriasis, mucosal psoriasis or ungual psoriasis, psoriatic rheumatism, cutaneous atopy, eczema, respiratory atopy or gingival hypertrophy; dermal or epidermal proliferations, benign or malignant, of viral or other origin, common warts, flat warts, verruciform epidermodysplasia, oral or florid papillomatoses and T lymphoma; proliferations induced by ultraviolet radiation, basal cell epithelioma and spinocellular epithelioma; precancerous skin lesions, keratoacanthomas; immune dermatoses, lupus erythematosus; bullous immune diseases; collagen diseases, scleroderma; dermatological or systemic disorders having an immunological component; skin disorders due to exposure to UV radiation, skin aging, light-induced or chronological, or actinic keratoses and pigmentations, or any pathologies associated with chronological aging or actinic aging, xerosis; sebaceous function disorders, hyperseborrhoea of acne, simple seborrhoea or seborrhoic dermatitis; cicatrization disorders or stretchmarks; pigmentation disorders, hyperpigmentation, melasma, hypopigmentation or vitiligo; lipid metabolism ailments disorders, obesity, hyperlipidemia, non-insulin-dependent diabetes or syndrome X; inflammatory disorders, arthritis; cancerous or precancerous conditions; alopecia of various origins, alopecia due to chemotherapy or to radiation; immune system disorders, asthma, type I sugar diabetes, multiple sclerosis, or other selective dysfunctions of the immune system; or disorders of the cardiovascular system, arteriosclerosis or hypertension, comprising spraying onto the affected skin area of an individual in need of such treatment, a thus effective amount of the sprayable, anhydrous dermatological/pharmaceutical composition as defined by claim 1.

15. A regime or regimen for the treatment of psoriasis, comprising spraying onto the affected area of the skin of an individual afflicted with psoriasis, of the sprayable, anhydrous dermatological/pharmaceutical composition as defined by claim 1.

16. A spray dispenser comprising a housing confining a sprayable, anhydrous dermatological/pharmaceutical composition as defined by claim 1, and a pumping element for mechanically spraying said composition out of said housing.

17. A spray dispenser comprising a housing confining a sprayable, anhydrous dermatological/pharmaceutical composition as defined by claim 1, and a gaseous propellant for spraying said composition out of said housing.

18. The spray dispenser as defined by claim 16, further comprising a metering element for spraying/delivering essentially the same amount of said composition.

19. The spray dispenser as defined by claim 17, comprising an amount of propellant effective for spraying/delivering essentially the same amount of said composition.
